(19) United States
(12) Patent Application Publication
(10) Pub. No.: US 2024/0350508 A1
(43) Pub. Date: Oct. 24, 2024

(54) USE OF EZH2 INHIBITORS FOR THE TREATMENT OF AORTIC VALVE STENOSIS

(71) Applicants: INSERM (INSTITUT NATIONAL DE LA SANTÉ ET DE LA RECHERCHE MÉDICALE), Paris (FR); CENTRE HOSPITALIER UNIVERSITAIRE DE ROUEN, Rouen (FR); UNIVERSITÉ DE ROUEN NORMANDIE, Mont-Saint-Aignan (FR)

(72) Inventors: Sylvain FRAINEAU, Rouen (FR); Nicolas PERZO, Rouen (FR)

(21) Appl. No.: 18/683,083

(22) PCT Filed: Aug. 24, 2022
(86) PCT No.: PCT/EP2022/073607

§ 371 Date: Feb. 12, 2024

(30) Foreign Application Priority Data
Aug. 25, 2021 (EP) 21306150.0

(51) Int. Cl. A61K 31/5377; A61F 2/24; A61K 31/496; A61P 9/10

(52) U.S. Cl. CPC A61K 31/5377; A61F 2/24; A61K 31/496; A61P 9/10; A61F 2250/0067

(57) ABSTRACT

Aortic valve stenosis (AS) also called also called Calcific aortic valve disease (CAVD), is the most frequent valvular heart disease in Europe and affects more than 1 in 4 people over 65 years old. AS progression from fibrotic thickening to valvular leaflets calcification leads to heart failure development and eventually to death within 2 to 5 years after symptoms occurrence. The inventors now show that EZH2 inhibition with GSK-126 and GSK-343 directly regulates monocyte and M1 toward M2 macrophage differentiation, reducing VIC deactivation and osteoblastic transition and thus represents an attractive therapeutic target to prevent AS progression. Therefore, the present invention relates to use of EZH2 inhibitors for the treatment of aortic valve stenosis.

### FIELD OF THE INVENTION

The present invention is in the field of medicine, in particular cardiology.

### BACKGROUND OF THE INVENTION

Aortic valve stenosis (AS) also called Calcific aortic valve disease (CAVD), is the most frequent valvular heart disease in Europe and affects more than 12.4% of population over 75 years old 3.5% of them presenting a severe form. CAVD progression from fibrotic thickening to valvular leaflets calcification leads to heart failure development and eventually to death within 2 to 5 years after symptoms occurrence. Its prevalence increases due to rapid aging of population and will become a public health issue within the next years. Up to date there are no pharmacological treatments for this pathology, CAVD patients being currently treated only with surgical or transcatheter aortic valve replacement (TAVI). Unfortunately, many patients are not eligible for these procedures due to a wide panel of associated co-morbidities, highlighting the critical and urgent need to develop nonsurgical pharmacological treatments.

CAVD initiation is due to Valvular Endothelial Cells (VECs), forming the upper layer of valve leaflets, dysfunction promoting monocytes recruitment (Morvan, Arangalage et al. 2019). Recruited monocytes will further undergo differentiation into M1 pro-inflammatory monocytes secreting pro-inflammatory cytokines responsible for inner leaflets Valvular Interstitial Cells (VICs) deactivation (Grim, Aguado et al. 2020) and further differentiation into osteoblast-like cells (Li, Qiao et al. 2017, Raddatz, Huffstater et al. 2020). This phenomenon is responsible for the production of micro and macro-calcification increasing valvular aortic leaflets rigidity and subsequent gradual decrease of the aortic orifice opening. CAVD progression is due to the imbalance between recruited M1 monocytes-derived pro-inflammatory macrophages and M2 resident immunomodulatory macrophages populations in the valvular leaflets (Hulin, Hego et al. 2018, Hulin, Hortells et al. 2019, Hénaut, Candellier et al. 2019, Karadimou, Plunde et al. 2020). Myeloid cells recruitment (monocytes) and differentiation (into M1 macrophages) represents a critical corner stone during CAVD progression.

In this context, macrophage phenotype switching, from M1 to M2-like, represents an attractive therapeutic target to design innovative treatments to prevent or cure CAVD patients. Accumulating evidences incriminate histone H3 lysine 27 trimethylation (H3K27me3) epigenetic histone modification, as a critical mechanism regulating macrophage activation and polarization (Bowdridge and Gause 2010, Van den Bossche, Neele et al. 2014, Davis and Gallagher 2019). Enhancer of zeste homolog 2 (EZH2) part of the Polycomb repressive complex 2 (PRC2) is responsible for generating the H3K27me3 epigenetic modification. We (Rondeaux et al., under review at Nat Com) and others have demonstrated that EZH2 localization and/or activity is responsible for macrophage activation by suppressing the expression of anti-inflammatory genes (Qiao, Kang et al. 2016, Neele and de Winther 2018, Zhang, Wang et al. 2018) while H3K27me3 demethylation enzymes and EZH2 antagonistic partners are required for M2 macrophage polarization after helminth infection (Satoh, Takeuchi et al. 2010) and IL4 stimulation in both human monocytes (Hsu, Lupancu et al. 2018) and murine macrophages (Ishii, Wen et al. 2009).

Altogether, these findings suggest that pharmacological inhibition of EZH2 activity might promote the switch from monocytes and M1 macrophages toward M2 macrophages phenotype preventing pro-inflammatory imbalance and subsequent VICs deactivation and differentiation into osteoblast-like cells. Thereby, GSK-126 and GSK-343, EZH2 inhibitors have emerged as potential candidate molecules given that they have already been shown to be effective in the treatment of various cancers (Béguelin, Popovic et al. 2013, Bitler, Fatkhutdinov et al. 2015), atherosclerosis (Wei, Zhang et al. 2021), coronary heart diseases (Liu, Dai et al. 2020), Transverse Aortic Constriction (TAC) and cardiac hypertrophy (Wang, Zhang et al. 2016, Shi, Fang et al. 2018), atrial fibrosis and fibrillation (Song, Zhang et al. 2019) or myocardial infarction (Rondeaux et al., under review at Nat Com).

### SUMMARY OF THE INVENTION

The present invention is defined by the claims. In particular, the present invention relates to use of EZH2 inhibitors for the treatment of aortic valve stenosis.

### DETAILED DESCRIPTION OF THE INVENTION

The inventors show that EZH2 inhibition with GSK-126 and GSK-343 directly regulates monocyte and M1 toward M2 macrophage differentiation, reducing VIC deactivation and osteoblastic transition and thus represents an attractive therapeutic target to prevent CAVD progression.

Accordingly, the first object of the present invention relates to a method of treating Aortic valve Stenosis (AS) in a patient in need comprising administering to the patient a therapeutically effective amount of an EZH2 inhibitor.

As used herein, the term “valve” may refer to the valve that prevents the backflow of blood during the rhythmic contractions. There are four main heart valves. In particular, the aortic valve separates the left ventricle and aorta. The term “aortic valve” includes a diseased aortic valve or a bioprosthetic valve.

As used herein, the term “bioprosthetic valve” is a stented tissue heart valve and may refer to a device used to replace or supplement an aortic valve that is defective, malfunctioning, or missing. Examples of bioprosthetic valve prostheses include, but are not limited to, TS 3fs® Aortic Bioprosthesis, Carpentier-Edwards PERIMOUNT Magna Ease Aortic Heart Valve, Carpentier-Edwards PERIMOUNT Magna Aortic Heart Valve, Carpentier-Edwards PERIMOUNT Magna Mitral Heart Valve, Carpentier-Edwards PERIMOUNT Aortic Heart Valve, Carpentier-Edwards PERIMOUNT Plus Mitral Heart Valve, Carpentier-Edwards PERIMOUNT Theon Aortic Heart Valve, Carpentier-Edwards PERIMOUNT Theon Mitral Replacement System, Carpentier-Edwards Aortic Porcine Bioprosthesis, Carpentier-Edwards Duraflex Low Pressure Porcine Mitral Bioprosthesis, Carpentier-Edwards Duraflex mitral bioprosthesis (porcine), Carpentier-Edwards Mitral Porcine Bioprosthesis, Carpentier-Edwards S.A.V. Aortic Porcine Bioprosthesis, Edwards Prima Plus Stentless Bioprosthesis, Edwards Sapien Transcatheter Heart Valve, Medtronic, Freestyle® Aortic Root Bioprosthesis, Hancock® II Stented Bioprosthesis, Hancock II Ultra® Bioprosthesis, Mosaic® Bioprosthesic, Mosaic Ultra® Bioprosthesis, St. Jude Medical, Biocor®, Biocor™ Supra, Biocor® Pericardia, Biocor™ Stentless, Epic™, Epic Supra™, Toronto Stentless Porcine Valve (SPV®), Toronto SPV II®, Trifecta, Sorin Group, Mitroflow Aortic Pericardial Valve®, Cryolife, Cryolife aortic Valve® Cryolife pulmonic Valve®, Cryolife-O'Brien stentless aortic xenograft Valve® and all variations thereof. Generally, bioprosthetic valve comprise a tissue valve having one or more cusps and the valve is mounted on a frame or stent, both of which are typically elastical. As used herein, the term “elastical” means that the device is able of flexing, collapsing, expanding, or a combination thereof. The cusps of the valve are generally made from tissue of mammals such as, without limitation, pigs (porcine), cows (bovine), horses, sheep, goats, monkeys, and humans.

As used herein, the term “stenosis” refers to the narrowing of the aortic valve that could block or obstruct blood flow from the heart and cause a back-up of flow and pressure in the heart.

As used herein, the term “aortic valve stenosis” or “AS” or “Calcific aortic valve disease” or “CAVD” has its general meaning in the art and refers to formation, growth or deposition of extracellular matrix hydroxyapatite (calcium phosphate) crystal deposits in the aortic valve.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In some embodiments, the method of the present invention is particularly suitable for primary prevention of AS. In particular, the method of the present invention is suitable for the treatment of calcific aortic valve disease. As used herein, the term “calcific aortic valve disease” or “CAVD” has its general meaning in the art and refers to a slowly progressive disorder with a disease continuum that ranges from mild valve thickening without obstruction of blood flow, termed aortic sclerosis, to severe calcification with impaired leaflet motion, or aortic stenosis.

Thus disease progression is generally characterized by a process of thickening of the valve leaflets and the formation of calcium nodules-often including the formation of actual bone—and new blood vessels, which are concentrated near the aortic surface. End-stage disease, e.g., calcific aortic stenosis, is generally characterized pathologically by large nodular calcific masses within the aortic cusps that protrude along the aortic surface into the sinuses of Valsalva, interfering with opening of the cusps. In some embodiments, the method of the present invention is particularly suitable for the treatment of calcific aortic stenosis.

In some embodiments, the method of the present invention is particularly suitable for secondary prevention of aortic valve stenosis. In particular, the method of the present invention is particularly suitable for preventing degeneration of an implanted bioprosthetic valve. Thus in some embodiment, the method of the present invention is particularly suitable for delaying or preventing the calcification of a bioprosthetic valve after valve replacement either surgically or after Transcatheter Aortic Valve Implantation (TAVI).

As used herein, the term “EZH2” has its general meaning in the art and refers to the enhancer of zeste homolog 2 encoded by the human EZH2 gene (Cardoso, C, et al; European J of Human Genetics, Vol. 8, No. 3 Pages 174-180, 2000). EZH2 is the catalytic subunit of the Polycomb Repressor Complex 2 (PRC2) which functions to silence target genes by tri-methylating lysine 27 of histone H3 (H3K27me3). Tri-methylation of H3K27 (H3K27me3) induces chromatin condensation and transcriptional repression of genes involved in development and differentiation (Bracken et al., 2006; Cao et al., 2002; Kirmizis et al., 2004).

As used herein, the term “EZH2 inhibitor” refers to a molecule that partially or fully blocks, inhibits, or neutralizes a biological activity or expression of EZH2. The EZH2 inhibitor can be a molecule of any type that interferes with EZH2 in a cell, for example, either by decreasing transcription or translation of EZH2-encoding nucleic acid, or by inhibiting or blocking EZH2 activity, or both. Examples of EZH2 inhibitors include, but are not limited to, antisense polynucleotides, interfering RNAs, catalytic RNAs, RNA-DNA chimeras, EZH2-specific aptamers, anti-EZH2 antibodies, EZH2-binding fragments of anti-EZH2 antibodies, EZH2-binding small molecules, EZH2-binding peptides, and other polypeptides that specifically bind EZH2 (including, but not limited to, EZH2-binding fragments of one or more EZH2 ligands, optionally fused to one or more additional domains), such that the interaction between the EZH2 inhibitor and EZH2 results in a reduction or cessation of EZH2 activity or expression.

EZH2 inhibitors are well known in the art (see e.g. Stazi G, Zwergel C, Mai A, Valente S. EZH2 inhibitors: a patent review (2014-2016). Expert Opin Ther Pat. 2017 July; 27 (7): 797-813. doi: 10.1080/13543776.2017.1316976. Epub 2017 Apr. 20. PMID: 28394193; Dockerill M, Gregson C, O′ Donovan D H. Targeting PRC2 for the treatment of cancer: an updated patent review (2016-2020). Expert Opin Ther Pat. 2021 February; 31 (2): 119-135. doi: 10.1080/13543776.2021.1841167. Epub 2021 Jan. 4. PMID: 33103538; Campbell J E, Kuntz K W, Knutson S K, Warholic N M, Keilhack H, Wigle T J, Raimondi A, Klaus C R, Rioux N, Yokoi A, Kawano S, Minoshima Y, Choi H W, Porter Scott M, Waters N J, Smith J J, Chesworth R, Moyer M P, Copeland R A. EPZ011989, A Potent, Orally-Available EZH2 Inhibitor with Robust in Vivo Activity. ACS Med Chem Lett. 2015 Mar. 4; 6 (5): 491-5. doi: 10.1021/acsmedchemlett.5b00037. PMID: 26005520; PMCID: PMC4434464; Li C, Wang Y, Gong Y, Zhang T, Huang J, Tan Z, Xue L. Finding an easy way to harmonize: a review of advances in clinical research and combination strategies of EZH2 inhibitors. Clin Epigenetics. 2021 Mar. 24; 13 (1): 62. doi: 10.1186/s13148-021-01045-1. PMID: 33761979; PMCID: PMC7992945; Hanaki S, Shimada M. Targeting EZH2 as cancer therapy. J Biochem. 2021 Jan. 21: mvab007. doi: 10.1093/jb/mvab007. Epub ahead of print. PMID: 33479735; Duan R, Du W, Guo W. EZH2: a novel target for cancer treatment. J Hematol Oncol. 2020 Jul. 28; 13 (1): 104. doi: 10.1186/s13045-020-00937-8. PMID: 32723346; PMCID: PMC7385862; Genta S, Pirosa M C, Stathis A. BET and EZH2 Inhibitors: Novel Approaches for Targeting Cancer. Curr Oncol Rep. 2019 Feb. 4; 21 (2): 13. doi: 10.1007/s11912-019-0762-x. PMID: 30715616). For example, non-limiting examples of EZH2 inhibitors include S-Adenosyl-Methionine (SAM)-competitive small molecule inhibitors. In some embodiments, the EZH2 inhibitor is derived from tetramethylpiperidinyl compounds. Further non-limiting examples of EZH2 inhibitors are described in Garapaty-Rao et al., Chemistry and Biology, 20: pp. 1-11 (2013), PCT Patent Application Nos. WO2003070887, WO2011140324, WO2011140325, WO2012075080, WO2012118812, WO2013/049770, WO2013138361, WO2015104677, WO2015110999, and US Patent Application Nos. US 2014/0275081, US 2012/0071418, US 2014/0128393 and US 2011/0251216, the contents of which are hereby incorporated by reference in their entireties. Further non-limiting examples include Tazemetaostat, UNC1999, 3-Deazaneplanocin A (DZNcp), EI1, EPZ-5676, EPZ-6438, GSK-343, EPZ005687, EPZ011989 and GSK-126.

In some embodiments, the EZH2 inhibitor is Tazemetostat (EPZ-6438) (CAS No.: 1403254-99-8) having the formula of:

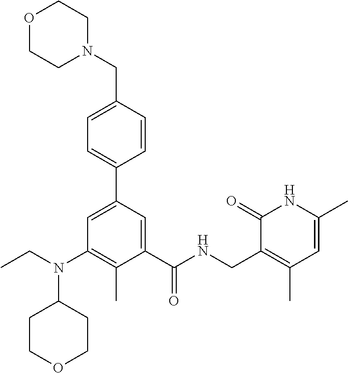 [SMILES: CCN(c1cc(-c2ccc(CN3CCOCC3)cc2)cc(C(=O)NCc2c(C)cc(C)[nH]c2=O)c1C)C1CCOCC1]

In some embodiments, the EZH2 inhibitor is GSK-126 ((GSK2816126A) (CAS No.: 1346574-57-9) having the formula of:

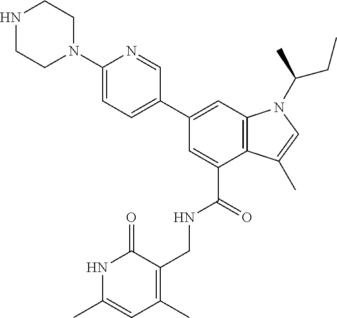 [SMILES: CC[C@H](C)n1cc(C)c2c(C(=O)NCc3c(C)cc(C)[nH]c3=O)cc(-c3ccc(N4CCNCC4)nc3)cc21]

In some embodiments, the EZH2 inhibitor is GSK-343 (CAS No.: 1346704-33-3) having the formula of:

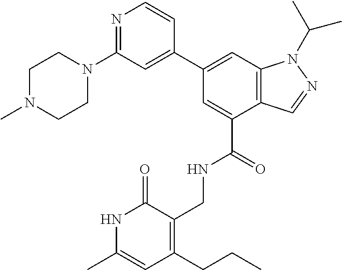 [SMILES: CCCc1cc(C)[nH]c(=O)c1CNC(=O)c1cc(-c2ccnc(N3CCN(C)CC3)c2)cc2c1cnn2C(C)C]

Further non-limiting examples of EZH2 inhibitors include ribozymes, antisense oligonucleotides, shRNA molecules and siRNA molecules that specifically inhibit the expression or activity of EZH2. One non-limiting example of an EZH2 inhibitor comprises an antisense, shRNA, or siRNA nucleic acid sequence homologous to at least a portion of a EZH2 nucleic acid sequence, wherein the homology of the portion relative to the EZH2 sequence is at least about 75 or at least about 80 or at least about 85 or at least about 90 or at least about 95 or at least about 98 percent, where percent homology can be determined by, for example, BLAST or FASTA software. In certain non-limiting embodiments, the complementary portion may constitute at least 10 nucleotides or at least 5 nucleotides or at least 20 nucleotides or at least 25 nucleotides or at least 30 nucleotides and the antisense nucleic acid, shRNA or siRNA molecules may be up to 15 or up to 20 or up to 25 or up to 30 or up to 35 or up to 40 or up to 45 or up to 50 or up to 75 or up to 100 nucleotides in length. Antisense, shRNA or siRNA molecules may comprise DNA or atypical or non-naturally occurring residues, for example, but not limited to, phosphorothioate residues.

As used herein, the term “therapeutically effective amount” is meant a sufficient amount of the EZH2 inhibitor for the treatment of the aortic valve stenosis at a reasonable benefit/risk ratio applicable to any medical treatment. It will be understood that the total daily usage of the compound will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the EZH2 inhibitor may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. The EZH2 inhibitor can be formulated into a composition in a neutral or salt form. Pharmaceutically acceptable salts include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils.

The present invention also relates to the use of an EZH2 inhibitor for the preparation of bioprosthetic valve. In this respect, the invention relates more particularly to bioprosthetic valve comprising an amount of an EZH2 inhibitor. Such a local biomaterial or medical delivery device can be used to treat aortic valve stenosis. Such biomaterial or medical delivery device may be biodegradable. The EZH2 inhibitor is preferably entrapped into the cusps of the valve. The cusps of the valve are generally made from tissue of mammals such as, without limitation, pigs (porcine), cows (bovine), horses, sheep, goats, monkeys, and humans. With said entrapment, it is possible to achieve a high level of local action. According to the present invention, the valve may be a collapsible elastical valve having one or more cusps and the collapsible elastical valve may be mounted on an elastical stent. In some embodiments, the collapsible elastical valve may comprise one or more cusps of biological origin. In some embodiments, the one or more cusps are porcine, bovine, or human. The elastical stent portion of the valve prosthesis used in the present invention may be self-expandable or expandable by way of a balloon catheter. The elastical stent may comprise any biocompatible material known to those of ordinary skill in the art. Examples of biocompatible materials include, but are not limited to, ceramics; polymers; stainless steel; titanium; nickel-titanium alloy, such as nitinol; tantalum; alloys containing cobalt, such as Elgiloy® and Phynox®; and the like. The process of disposing the coating composition which comprises the EZH2 inhibitor may be any process known in the art. The local delivery according to the present invention allows for high concentration of the EZH2 inhibitor at the disease site with low concentration of circulating compound. For purposes of the invention, a therapeutically effective amount will be administered.

The invention will be further illustrated by the following figures and examples. However, these examples and figures should not be interpreted in any way as limiting the scope of the present invention.

### FIGURES

FIG. 1: Ezh2 inhibition reduces human volunteer monocytes secretome-induced VICs calcification. Data are represented as mean values±SEM (n=4 VICs patients in triplicates, CM1 from n=3 human volunteers). Asterisk (*) symbols indicates statistically significant difference compared to vehicle (DMSO) condition after Kruskal-Wallis test. * p<0.05, ** p<0.01, *** p<0.001.

FIG. 2: Ezh2 inhibition reduces human CAVD patient monocytes secretome-induced VICs calcification. Data are represented as mean values±SEM (n=6 VICs patients in triplicates, CM1 from n=3 human CAVD patients). Asterisk (*) symbols indicates statistically significant difference compared to vehicle (DMSO) condition after Kruskal-Wallis test. ** p<0.01, *** p<0.001.

FIG. 3: Ezh2 inhibition reduces human volunteer monocytes secretome-induced VICs calcification 24 h after monocytes pre-treatment. Data are represented as mean values±SEM (n=5 VICs patients in triplicates, CM2 from n=3 human volunteers). Asterisk (*) symbols indicates statistically significant difference compared to vehicle (DMSO) condition after Kruskal-Wallis test. ** p<0.01.

FIG. 4: Ezh2 inhibition reduces human M1 macrophages secretome-induced VICs calcification (n=1 VICs patient in triplicates, CM from n=1 human volunteer)

### EXAMPLE

### Methods

Human Valvular Interstitial Cells (hVICs) Isolation and Culture

Human aortic valves form AS patients who underwent aortic valve surgical replacement were collected at Rouen University Hospital, starting on May 2 2016, after agreement of The CPP Nord-Ouest I (RCB: 2016-A00137-44). Human Valvular Interstitial Cells (hVICs) were isolated and cultured as already described. (Morvan, Arangalage et al. 2019) Briefly, aortic valve leaflets previously collected and washed in PBS containing antibiotics were cut into pieces and digested with type I collagenase (0.22 U/mg) for 3 hours at 37° C. Digested valves are filters on 70 μm cell strainers to remove undigested valve parts and obtain a homogenous cell suspension resuspended in complete smooth muscle cell basal 2 medium (PromoCell, Heidelberg, Germany) with 10% Fetal Bovine Serum (FBS) Penicillin/Streptomycin (P/S, Sigma-Aldrich Cat #P4333). Cell suspension is then seeded into rat type collagen I (3 mg/ml, Gibco, Cat #A1048301) coated flask and incubated at 37° C., 5% CO₂ for several days until reaching cell confluency. At this step hVICs are obtained and cultured in DMEM (Dulbecco's modified Eagle's medium, Gibco, Cat #41966-029) with 10% FBS and P/S until passage 4.

### Human Monocytes Isolation and Culture

Monocytes were directly isolated from either fresh human CAVD patients (Ref: 2021/229/OB, ID RCB: 2021-A01850-41) or volunteers pooled EDTA peripheral blood samples (Etablissement Français du Sang, Laboratoire Produits de Laboratoire Enseignement et Recherche, Bois Guillaume, France) by negative selection using EasySep™ monocyte isolation kits (StemCell Technologies Cat #19669, Vancouver, Canada) according to the manufacturer's instructions. Monocytes were then seeded at a cellular density >150 000 ¢/cm² and cultured in DMEM (Gibco, Cat #41966-029) supplemented with 50 μM of 2-mercaptoethanol (Gibco Cat #21985-023), 10% FBS (Gibco Cat #10500-064), Penicillin/Streptomycin (P/S) and hMacrophage-Colony Stimulating Factor (hM-CSF, 50 ng/ml, StemCell Technologies Cat #78057.1, Vancouver, Canada). Monocytes were differentiated into M0 macrophages after incubation for 4 days with 50 ng/ml hM-CSF. M0 macrophages were further polarized into M1 macrophages after treatment with 50 ng/ml of LipoPolySaccharide (LPS, Sigma-Aldrich Cat #L6529) for 2 days. Selected monocytes and M1 macrophages were then treated for 72 h in the presence of 5 μM EZH2 inhibitors GSK-126 (MedChemExpress, Cat #HY-13470, Monmouth Junction, USA) and GSK-343 (Sigma Aldrich, Cat #SML0766, Saint-Louis, USA) or DiMethyl SulfOxide (DMSO, vehicle). Conditioned media (CM) were harvested either immediately after monocytes pre-treatment (CM1) or after 24 h incubation in DMEM (Gibco, Cat #41966-029) supplemented with 50 μM of 2-mercaptoethanol (Gibco Cat #21985-023), 10% FBS (Gibco Cat #10500-064) and P/S at 37° C., 5% CO₂ following monocytes pre-treatment or M1 macrophages differentiation (CM2).

hVICs Calcification Measurement

Previously isolated hVICs were treated for 10 days with human monocytes and M1 macrophages CM diluted v/v with DMEM (Gibco, Cat #41966-029) in the presence or not of inorganic PyroPhosphate (Pi) at a final concentration up to 1.9 mM (0.9 mM of PPi already present in DMEM). Treatment with conditioned medium was renewed every 2 days and calcium content in each well was measured with the o-cresolphthalein complexone colorimetric method. (Louvet, Büchel et al. 2013, Varennes, Mentaverri et al. 2020)

### Statistical Analyses

Data are obtained from indicated number of hVICs isolated from distinct patients. Each o-cresolphthalein complexone measurement experiment was performed in triplicate. Data are expressed as mean values of control (DMSO (vehicle) treated monocytes or M1 macrophages)±SEM. When indicated, statistical significance was determined by non-parametric Kruskal-Wallis test followed by Dunns post-hoc test using Graphpad Prism 5 software. The use of non-parametric Kruskal-Wallis test was determined depending Shapiro-Wilk normality test data distribution. Data are considered to be significantly different at values p<0.05.

### Results

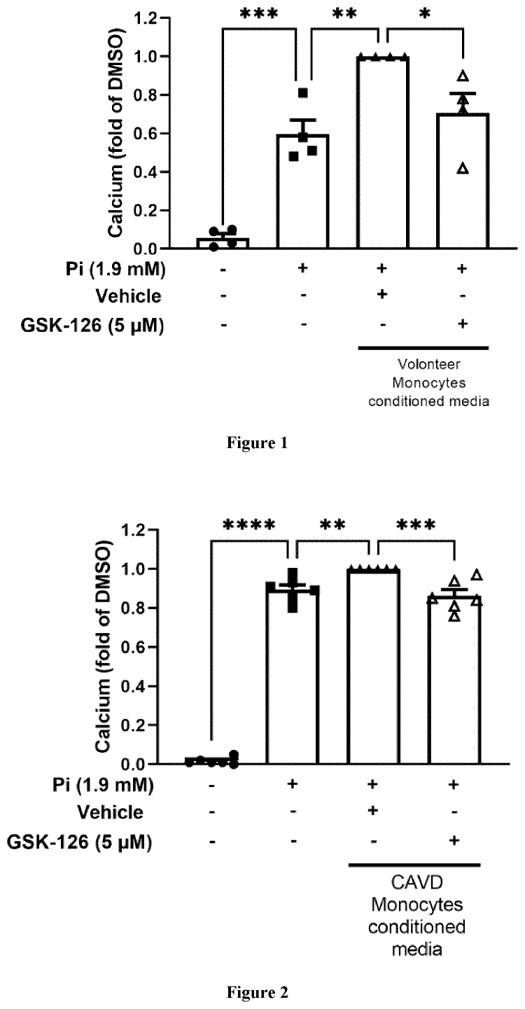

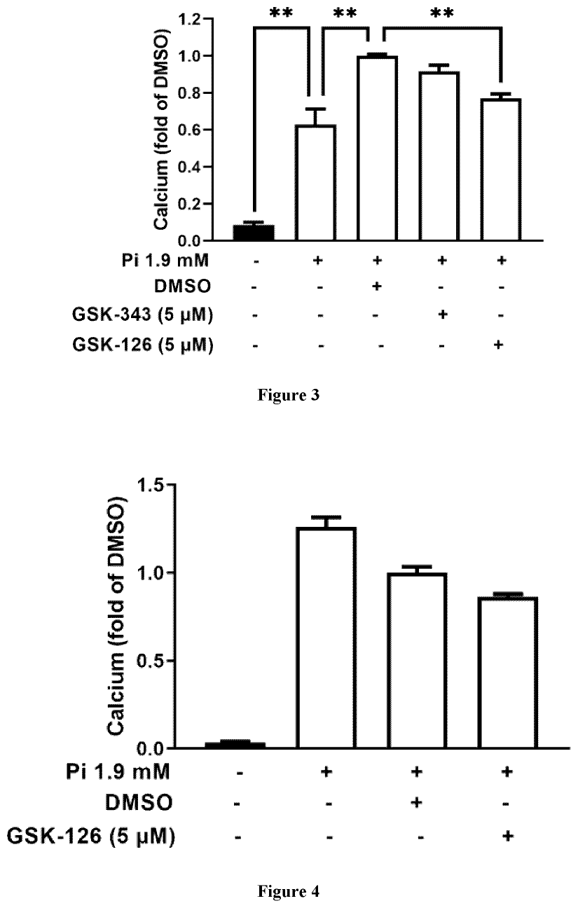

We first investigated the role of Ezh2 in human monocytes secretome-induced hVICs calcification. As expected, we observed a calcification of hVICs in the presence of 1.9 mM Pi (FIGS. 1, 2 and 3). This calcification was further significantly potentiated by human monocytes secretome (vehicle condition) conditioned for 72 h during pre-treatment (CM1) isolated from volunteers (FIG. 1) or CAVD patients (FIG. 2) peripheral blood immediately. Pre-treatment of monocytes with EZH2 inhibitor GSK-126 (5 μM) for 72 h significantly reduced volunteer (FIG. 1) and CAVD patient (FIG. 2) monocytes secretome-induced calcification. Interestingly, the same result was obtained after allowing monocytes isolated from volunteers peripheral blood to produce a new conditioned medium for 24 h (CM2) in the absence of EZH2 inhibitor, GSK-126, following the 72 h pre-treatment (FIG. 3). These data suggest that GSK-126 might be an interesting therapeutic target to prevent monocytes dependent VICs calcification during AS development.

To better assess the potential EZH2 inhibitors in pro-inflammatory myeloid cells secretome-induced VICs calcification, we differentiated human selected monocytes into M0 macrophages prior to their polarization into pro-inflammatory M1 macrophages well-known for their pro-calcifying activity on human VICs. As previously observed with human monocytes, M1 macrophages pre-treatment with GSK-126 reduced M1 secretome-induced hVICs calcification (FIG. 4).

Thus, EZH2 inhibitor GSK-126, is an interesting therapeutic epigenetic treatment to prevent both monocytes and M1 macrophages secretome-induced hVICs calcification and subsequent AS development. We believe that this treatment is of interest to prevent AS development in patients.

### REFERENCES

Throughout this application, various references describe the state of the art to which this invention pertains. The disclosures of these references are hereby incorporated by reference into the present disclosure.
Bitler, B. G., et al. (2015). “Potential therapeutic targets in ARIDIA-mutated cancers.” Expert Opin Ther Targets 19 (11): 1419-1422.
Bowdridge, S. and W. C. Gause (2010). “Regulation of alternative macrophage activation by chromatin remodeling.” Nat Immunol 11 (10): 879-881.
Béguelin, W., et al. (2013). “EZH2 is required for germinal center formation and somatic EZH2 mutations promote lymphoid transformation.” Cancer Cell 23 (5): 677-692.
Davis, F. M. and K. A. Gallagher (2019). “Epigenetic Mechanisms in Monocytes/Macrophages Regulate Inflammation in Cardiometabolic and Vascular Disease.” Arterioscler Thromb Vasc Biol 39 (4): 623-634.
Grim, J. C., et al. (2020). “Secreted Factors From Proinflammatory Macrophages Promote an Osteoblast-Like Phenotype in Valvular Interstitial Cells.” Arterioscler Thromb Vasc Biol 40 (11): e296-e308.
Hsu, A. T., et al. (2018). “Epigenetic and transcriptional regulation of IL4-induced CCL17 production in human monocytes and murine macrophages.” J Biol Chem 293 (29): 11415-11423.
Hulin, A., et al. (2018). “Advances in Pathophysiology of Calcific Aortic Valve Disease Propose Novel Molecular Therapeutic Targets.” Front Cardiovasc Med 5:21.
Hulin, A., et al. (2019). “Maturation of heart valve cell populations during postnatal remodeling.” Development 146 (12).
Hénaut, L., et al. (2019). “New Insights into the Roles of Monocytes/Macrophages in Cardiovascular Calcification Associated with Chronic Kidney Disease.” Toxins (Basel) 11 (9).
Ishii, M., et al. (2009). “Epigenetic regulation of the alternatively activated macrophage phenotype.” Blood 114 (15): 3244-3254.
Karadimou, G., et al. (2020). “TLR7 Expression Is Associated with M2 Macrophage Subset in Calcific Aortic Valve Stenosis.” Cells 9 (7).
Li, G., et al. (2017). “The shift of macrophages toward M1 phenotype promotes aortic valvular calcification.” J Thorac Cardiovasc Surg 153 (6): 1318-1327.e1311.
Liu, Y., et al. (2020). “Inhibition of EZH2 attenuates coronary heart disease by interacting with microRNA-22 to regulate the TXNIP/nuclear factor-KB pathway.” Exp Physiol 105 (12): 2038-2050.
Louvet, L., et al. (2013). “Magnesium prevents phosphate-induced calcification in human aortic vascular smooth muscle cells.” Nephrol Dial Transplant 28 (4): 869-878.
Morvan, M., et al. (2019). “Relationship of Iron Deposition to Calcium Deposition in Human Aortic Valve Leaflets.” J Am Coll Cardiol 73 (9): 1043-1054.
Neele, A. E. and M. P. J. de Winther (2018). “Repressing the repressor: Ezh2 mediates macrophage activation.” J Exp Med 215 (5): 1269-1271.
Qiao, Y., et al. (2016). “IFN-γ Induces Histone 3 Lysine 27 Trimethylation in a Small Subset of Promoters to Stably Silence Gene Expression in Human Macrophages.” Cell Rep 16 (12): 3121-3129.
Raddatz, M. A., et al. (2020). “Macrophages Promote Aortic Valve Cell Calcification and Alter STAT3 Splicing.” Arterioscler Thromb Vasc Biol 40 (6): e153-e165.
Satoh, T., et al. (2010). “The Jmjd3-Irf4 axis regulates M2 macrophage polarization and host responses against helminth infection.” Nat Immunol 11 (10): 936-944.
Shi, Z. L., et al. (2018). “EZH2 Inhibition Ameliorates Transverse Aortic Constriction-Induced Pulmonary Arterial Hypertension in Mice.” Can Respir J 2018:9174926.
Song, S., et al. (2019). “EZH2 as a novel therapeutic target for atrial fibrosis and atrial fibrillation.” J Mol Cell Cardiol 135:119-133.
Van den Bossche, J., et al. (2014). “Macrophage polarization: the epigenetic point of view.” Curr Opin Lipidol 25 (5): 367-373.
Varennes, O., et al. (2020). “The Metabolism of Epoxyeicosatrienoic Acids by Soluble Epoxide Hydrolase Is Protective against the Development of Vascular Calcification.” Int J Mol Sci 21 (12).
Wang, Z., et al. (2016). “The long noncoding RNA Chaer defines an epigenetic checkpoint in cardiac hypertrophy.” Nat Med 22 (10): 1131-1139.
Wei, X., et al. (2021). “Pharmacological inhibition of EZH2 by GSK126 decreases atherosclerosis by modulating foam cell formation and monocyte adhesion in apolipoprotein E-deficient mice.” Exp Ther Med 22 (2): 841.
Zhang, X., et al. (2018). “Macrophage/microglial Ezh2 facilitates autoimmune inflammation through inhibition of Socs3.” J Exp Med 215 (5): 1365-1382.

1. A method of treating Aortic valve Stenosis (AS) in a patient in need thereof comprising administering to the patient a therapeutically effective amount of an EZH2 inhibitor.

2. The method of claim 1 wherein the patient suffers from a calcific aortic valve disease.

3. The method of claim 1 wherein administration of the EZH2 inhibitor prevents degeneration of an implanted bioprosthetic valve.

4. The method of claim 3 wherein administration of the EZH2 inhibitor delays or prevents calcification of a bioprosthetic valve after valve replacement either surgically or after transcatheter aortic valve implantation (TAVI).

5. The method of claim 1 wherein the EZH2 inhibitor is selected from the group consisting of Tazemetostat, GSK-126 and GSK-343.

6. A method of preparing a bioprosthetic valve, comprising
during preparation of the bioprosthetic valve, entrapping an EZH2 inhibitor in cusps of the bioprosthetic valve.

7. A bioprosthetic valve comprising an amount of an EZH2 inhibitor.

8. The bioprosthetic valve of claim 7 wherein the EZH2 inhibitor is entrapped the within cusps of the bioprosthetic valve.

9. A method of preventing or treating aortic valve stenosis in a subject in need thereof, comprising, implanting the bioprosthetic valve of claim 7 into the subject.
